(19) United States
(12) Patent Application Publication
(10) Pub. No.: US 2019/0298570 A1
(43) Pub. Date: Oct. 3, 2019

(54) WARMING DEVICE AND METHOD

(71) Applicant: KVI LLC, EDEN PRAIRIE, MN (US)

(72) Inventors: Thomas P. Clement, Bloomington, IN (US); Alan Thomas Barker, Bloomington, IN (US)

(21) Appl. No.: 16/372,990

(22) Filed: Apr. 2, 2019

Related U.S. Application Data
(60) Provisional application No. 62/651,418, filed on Apr. 2, 2018.

(51) Int. Cl. A61F 7/12

(52) U.S. Cl. CPC A61F 7/12

(57) ABSTRACT

A flexible malleable warming device generates heat through an exothermic chemical reaction. The device can conform to an endoscope to heat it for use. The device can further include one or more surfaces for applying anti-fogging solution and white-balancing the camera of the endoscope.

### CROSS REFERENCE TO RELATED APPLICATION

This application claims the benefit of priority to U.S. Patent Provisional Application No. 62/651,418, filed Apr. 2, 2018, which is incorporated by reference herein in its entirety.

### BACKGROUND

Surgical processes often use endoscopes for viewing inside the body. Because of the temperature difference between a lens of the endoscope and the internal body temperature, the lens of the scope can fog up. Therefore, anti-fogging products such as solutions exist to mitigate this fogging.

Some anti-fogging solutions can be applied directly to the surface of the lens. However, these and other endoscope warming technologies can be cumbersome to manipulate whether due to a difficult heat activation process or the risk of spilling the solution. In addition, in order to achieve correct color-rendering in captured images, the camera of the scope must be white-balanced. This requires an additional step of using an extrinsic material as a color reference sample before use of the endoscope.

### SUMMARY

The present disclosure may comprise one or more of the following features and combinations thereof.

A flexible warming device may comprise a sealed pouch forming an interior lumen enclosing an exothermic reactant and a burstable pouch positioned within the interior lumen of the sealed pouch containing a catalyst. The catalyst mixes with the exothermic reactant to generate heat. The at least one pouch surface may be configured to transfer the generated heat within the pouch to an endoscope positioned exterior to and contacting pouch, to absorb anti-fogging solution and transfer it to the endoscope and provide a white-balancing color reference surface to a camera of the endoscope.

In some embodiments, the reactant comprises calcium oxide and the catalyst comprises an aqueous solution. In some embodiments the sealed pouch further comprises a rheological modifier or kosmotropic agent configured to control the rate of heat generation when the catalyst mixes with the exothermic reactant.

In some embodiments, the exothermic reactant does not produce a gaseous byproduct or a toxic product in response to mixing with the catalyst.

In some embodiments, the exothermic reactant comprises a powder mixture. The powder mixture may comprise a primary fast-reactant and a secondary reactant, wherein the secondary reactant reacts at a slower rate and reaches a peak heat generation with the catalyst later in time than the primary fast-reactant. The primary fast-reactant and secondary reactant may react with the catalyst to generate a heat output in a range of 105-180 degrees Fahrenheit.

In some embodiments, the primary fast-reactant and secondary reactant react with the catalyst to generate a heat output in a range of 105-180 degrees Fahrenheit and maintain the heat output in the range for 20-30 minutes.

In some embodiments, at least one pouch surface comprises a rubber material, a sorptive material, and the color-reference material is a white-balancing material. The at least one surface may be single surface on the device comprising heat-transferring, sorptive, and color-reference material properties. In some embodiments the at least one surface may be three distinct surfaces spaced from each other along the outside of the device.

According to another aspect of the present disclosure a device for warming an endoscope for insertion into a body may include a flexible and malleable pouch having a single sealed interior lumen, a dry mixture containing an exothermic reactant positioned in the interior lumen, and a burstable pouch positioned in the interior lumen containing an aqueous catalyst. The burstable pouch may be configured to break in response to application of an external force to the flexible and malleable pouch and release the aqueous catalyst to mix with the dry mixture and generate heat.

In some embodiments, the exothermic reactant comprises calcium oxide and the catalyst comprises an aqueous solution.

In some embodiments, the dry mixture comprises a primary fast-reactant and a secondary reactant, and the secondary reactant reacts at a slower rate with the catalyst and reaches a maximum temperature later in time than the primary fast-reactant. The primary fast-reactant and secondary reactant may react with the catalyst to generate a heat output in a range of 105-180 degrees Fahrenheit.

In some embodiments, the primary fast-reactant and secondary reactant react with the catalyst to generate a heat output in a range of 105-180 degrees Fahrenheit and maintain the heat output in the range for 20-30 minutes.

According to another aspect of the present disclosure a method for preparing an endoscope for use inside the body includes applying a force to a flexible warming device to release a catalyst from a rupturable pouch inside the device, massaging the flexible warming device to distribute the catalyst with a reactant in the flexible warming device thereby generating heat, and molding the flexible warming device to contact the endoscope, thereby transferring heat to the endoscope and providing contact between a lens of the endoscope and a color-reference surface of the endoscope.

In some embodiments, the method may further comprise applying an anti-fogging solution to the color-reference surface of the device and transferring the anti-fogging solution to the lens via the contact between the lens and the color-reference surface. The color reference surface may be a sorptive surface. The method may further include white-balancing a camera of the endoscope using the color-reference surface. The heat transferring, anti-fogging solution transferring, and white-balancing may be performed simultaneously.

In some embodiments, the catalyst comprises an aqueous solution and the reactant comprises a mixture of fast and slow-reacting powders.

These and other features of the present disclosure will become more apparent from the following description of the illustrative embodiments.

### BRIEF DESCRIPTION OF THE DRAWINGS

FIG. 1 is a perspective view of a warming device according to various embodiments.

FIG. 2 is a cross-sectional view of the warming device of FIG. 1 exposing an arrangement of contents in the interior of the warming device according to various embodiments.

FIG. 3 shows a method of using a warming device to prepare an endoscope to be used in a surgical procedure according to various embodiments.

FIGS. 4A-4B show perspective views of a warming device according to various embodiments.

FIG. 5 shows the performance of Example 1 and Example 2.

### DETAILED DESCRIPTION

For the purposes of promoting an understanding of the principles of the disclosure, reference will now be made to a number of illustrative embodiments illustrated in the drawings and specific language will be used to describe the same.

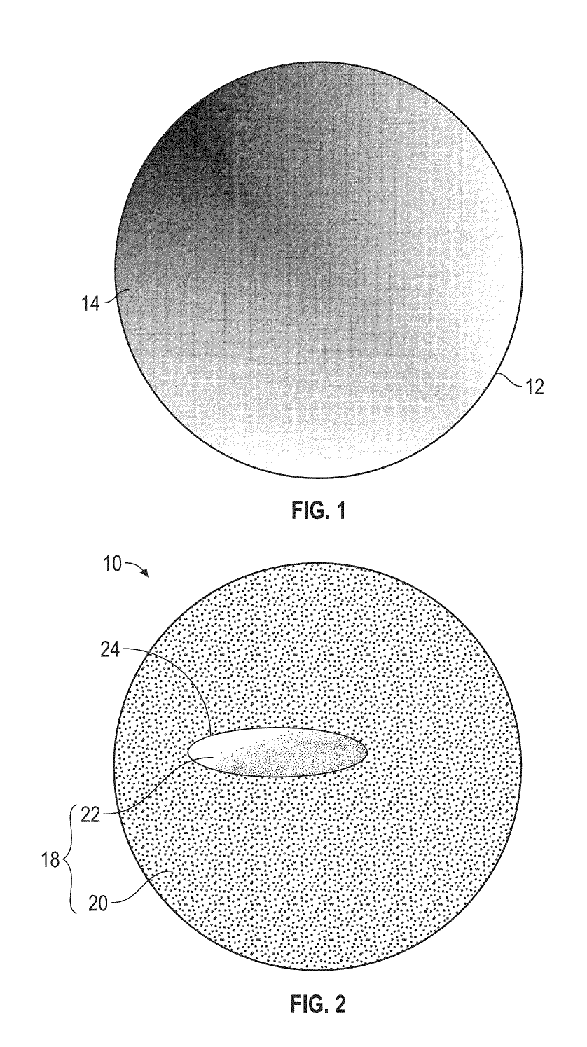

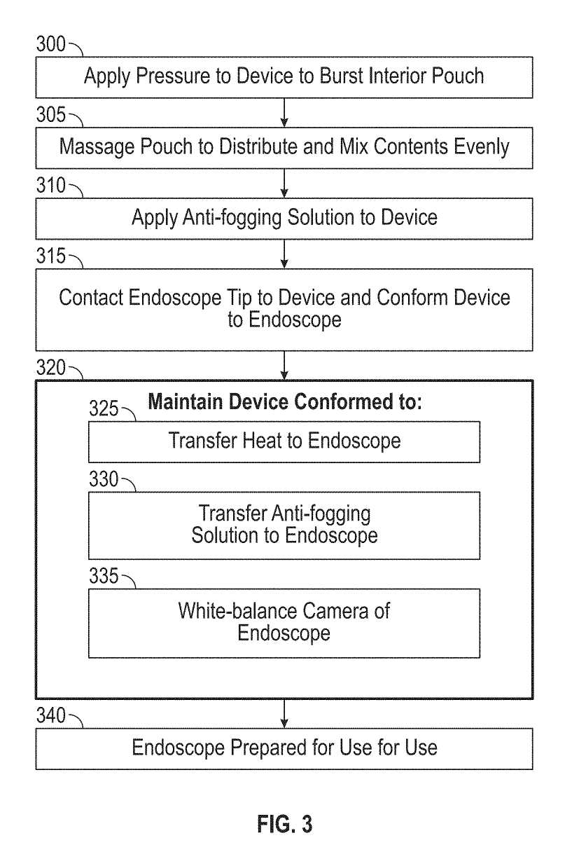

As seen in FIG. 1, a flexible warming device 10 is provided that is a spherical shaped pouch defining a sealed internal lumen. Warming device 10 is configured to be sufficiently flexible and malleable to conform to irregular surfaces, including a tip of an endoscope. The warming device includes at least one surface 12 and a second surface 14.

At least one surface 12 of the device 10 may be configured as a color-reference for white-balancing. The surface 12 may also be of a sorbent pad material that permits application of an anti-fogging solution to be transferred to the lens of the scope during the process of heating and white-balancing the scope. The material may be a mildly sorptive surface of cloth or microfiber that is charged with an anti-fogging solution that can be applied directed to the exposed surface of the device being warmed. At least one surface 14 of the flexible warming device 10 may be a heat-transferring surface that is formed of a thermally-conductive material. By way of example, this thermally conductive material may be a rubber material. Flexible warming device 10 may alternatively be disc or puck-shaped. In some embodiments the at least one surface providing the anti-fogging and white-balancing, and the thermally conductive surface may be the same surface of the warming device 10 so that heating, anti-fogging and white-balancing may be simultaneously executed.

Sealed internal lumen 14 of warming device 10 can be seen in a cross-section of the warming device 10 in FIG. 2. The lumen contains an exothermic chemically-based warming component 18 disposed therein. The chemical warming component 18 includes a liquid portion including a catalyst 22 and dry portion including a reactant 20. Catalyst 22 may be separated from the reactant 20 by a burstable pouch 24. Burstable pouch 24 may enclose the catalyst 22 prior to use of the warming device 10. In operation, application of a force, such as squeezing or massaging, to the warming device 10 breaks the burstable pouch 24 causing the catalyst 2 and reactant 20 to mix thereby generating heat and form a putty-like malleable product. The catalyst and reactant are selected to instantaneously achieve a temperature range of 105-180 degrees Fahrenheit (e.g., from 115 to 160 degrees Fahrenheit).

The exothermic mixture may reach the desired temperature range within two or three minutes and maintain the range for approximately 20-30 minutes. The exothermic mixture is also configured so that it produces no gases. Therefore, there is no need to vent the mixing reaction. The exothermic mixture is further configured so that it does not produce any byproducts that would result in toxicological hazards or require special waste disposal.

In an exemplary embodiment, the primary active constituents of the exothermic mixture are a reactant including two calcium salts present in controlled particle sizes and ratios which release heat upon exposure to a catalyst of water. Each of the two calcium salts may react in a different time-scale which when combined may provide consistent heating in a desired 20 to 30 minute time frame (e.g., from about 22 to 28 minutes).

According to one embodiment, a primary fast-reactant of calcium chloride, and a secondary reactant of calcium oxide are provided in a pouch in the dry portion of the exothermic mixture. The calcium chloride may be present both in a medium-fine mesh powder as well as pellets ranging in size from 1.0 mm up to approximately 3.0 mm (e.g., from about 1.5 mm to about 2.5 mm) present in roughly equal ratios to each other. As the calcium chloride exothermic reaction depletes, a reaction between the water in the pouch and a calcium oxide progresses, providing a higher-achievable temperature and extending the useful life of the warming device. The calcium oxide (CaO) used may be a milled powder and may be of two different forms, either alone, or present in various ratios to each other. One form is the fast-reactive form of CaO which releases a greater amount of heat in a shorter time-span, while the other is the slow-reactive form which reaches a lower peak temperature but reacts for a greater amount of time. These two forms of calcium oxide may be combined in some embodiments in order to achieve a quick rise to the peak temperature of the warming device, while also maximize operating time.

Other components in the dry portion of the exothermic mixture may include an inert hydrophilic substance. In some embodiments the inert hydrophilic substance may be comprised of microcrystalline cellulose, which serves to distribute water throughout the mixture, provide bulk mass for the heat-pack, prevent clumping of the reactants before and during activation, and aid in homogenous heat-distribution. In some embodiments, sodium lauryl sulfate (SDS) may also be included in the dry mixture at a rate of 1-5% the weight of the aqueous catalyst used. The SDS modulates the rate of reaction of the CaO with the water, slowing down its rate of temperature rise. Additionally, the surfactant properties of SDS aid in evenly distributing the water throughout the dry exothermic mixture.

Some embodiments may include an inert filler such as charcoal powder, silica perlite, or vermiculite. Some embodiments may also contain a gelling agent, or rheological modifier that controls and the rate of reaction by impeding the interfacing of the water with the exothermic reactants to slow the reaction. Examples include xanthan gum, guar gum, agar, alginate, and methylcellulose.

The liquid portion of the exothermic mixture may be an aqueous catalyst containing a kosmotropic agent which in some embodiments may illustratively be glycerol or propylene glycol in up to a 1:1 ratio with the water. The aqueous catalyst may be provided in an amount equaling approximately 80% of the weight of the dry reactants. The kosmotropic agent rate limits the reaction of the water with the CaO allowing the exothermic reaction to progress for a longer time period. Further it reduces the overall peak-temperature achieved by the system to a safe level in order to prevent harming the user in handling the pouch and prevents a build-up of steam within the pouch. Further examples of exothermic mixtures that may be used in the pouch are provided below.

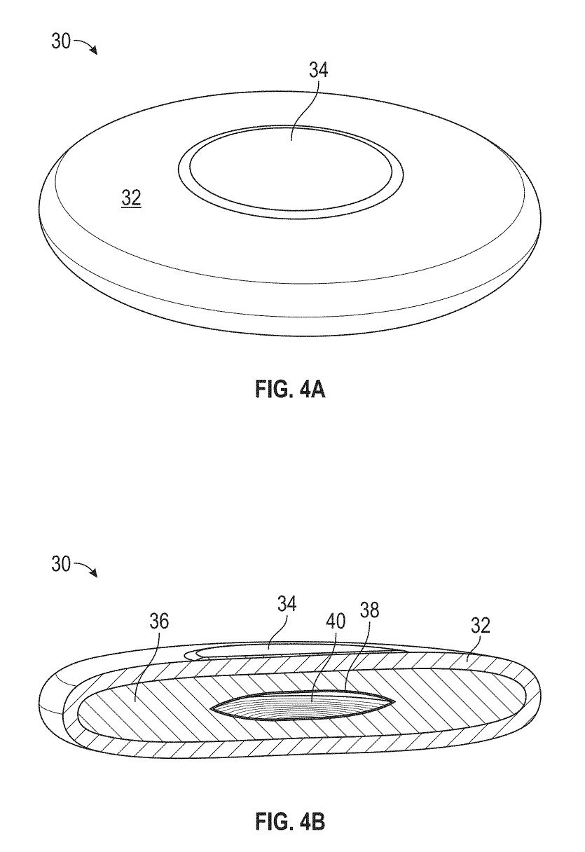

FIGS. 4A-4B show perspective views of a warming device according to various embodiments. FIG. 4A shows an external perspective view of a flexible warming device 30 that is a disk-like spherical shaped pouch defining a sealed internal lumen. FIG. 4B shows a cross-sectional view of the flexible warming device 30.

Flexible warming device 30 includes elastomeric enclosure 32 with white sorptive pad 34 residing thereon. Elastomeric enclosure 32 encircles powdered reactant 36 and burst pack 38 holding liquid catalyst 40. Burst pack 38 resides centrally within powdered reactant 36 and elastomeric enclosure 32.

As with device 10 of FIGS. 1-2, device 30 is a warming device for use with an endoscope. When in use, device 30 is inserted adjacent endoscope to prevent fogging and ensure warmth. When triggered, burst pack 38 bursts, mixing powdered reactant 36 with liquid catalyst 40 within elastomeric enclosure 32. This produces sustained heat that allows the endoscope to be warmed.

As discussed with reference to FIGS. 1 and 2, elastomeric enclosure 32 is a flexible material such as silicone, synthetic rubber, or other material appropriate in the art to stretch or deform when device 30 warmed prior to the endoscope being inserted into a body. Additionally, elastomeric enclosure 32 is made of a material that maintains its integrity when heated. Elastomeric enclosure 32 serves to hold together device 30 and ensure powdered reactant 36 and liquid catalyst 40 do not leak into a body while in use.

Sorptive pad 34 sits externally to elastomeric enclosure 32. Sorptive pad 34 can be used to align device 30 with an endoscope to prevent mis-alignment or mis-application. Sorptive pad 34 can optionally be coated in an anti-fogging solution to further prevent fogging of the endoscope. Additionally, sorptive pad 34 can be made of a reference white material to allow for white balance during application.

Powdered reactant 36 is a dry powder mix that is chemically reactive once exposed to liquid catalyst 40. Powdered reactant 36 can be, for example, a mixture of calcium oxide (CaO) and calcium chloride (CaCl₂) of varying forms (e.g., pellets or powder), in addition to microcrystalline cellulose.

Liquid catalyst 40 resides in sealed burst pack 38. Liquid catalyst interacts with the powdered reactant 36 to generate heat when burst pack 38 is broken. Liquid catalyst can include, for example, water, propylene glycol, and SDS. In this case, when the powdered reactant 36 reacts with the liquid catalyst 40, the reaction heats up quickly and maintains a workable temperature for the duration of the endoscope usage.

### Examples

Various embodiments of the present disclosure can be better understood by reference to the following Examples which are offered by way of illustration. The present disclosure is not limited to the Examples given herein.

Examples 1 and 2 were made of the mixtures show below in Table 1:

TABLE 1
Example 1 & 2

| | Dry Portion | | | | Aqueous Portion | | | |
|---|---|---|---|---|---|---|---|---|
| | | | | Micro- | | Pro- | | |
| | | CaCl₂ | CaCl₂ | crystalline | | pylene | Glyc- | |
| | CaO | Pellets | Powder | Cellulose | H₂O | Glycol | erol | SDS |
| | (g) | (g) | (g) | (g) | (mL) | (mL) | (mL) | (g) |
| 1 | 6.5 | 2.0 | 1.5 | 7.5 | 5 | 0 | 5 | 0.1 |
| 2 | 6.5 | 2.0 | 1.5 | 7.5 | 5 | 5 | 0 | 0.1 |

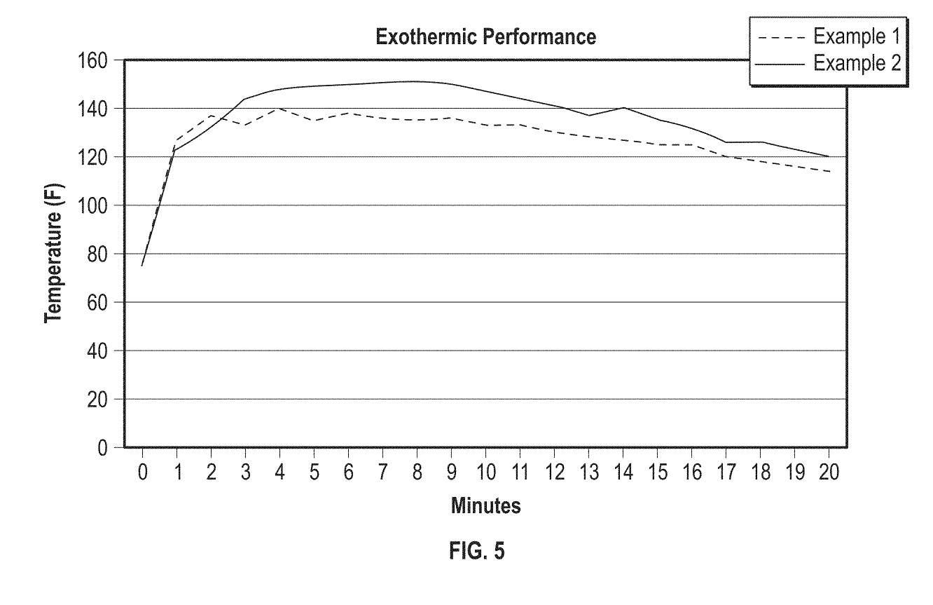

Examples 1 and 2 contain different kosmotropes; propylene glycol and glycerol respectively. FIG. 5 shows the performance of Example 1 and Example 2 as temperature (in degrees Fahrenheit) versus time (in minutes).

In an alternative embodiment the reactant may be a blend of iron powder, sodium chloride, and an inert filler and the catalyst may be an acidic solution. In some embodiments the acid in solution may be acetic acid. In some embodiments the acid may be phosphoric acid. In some embodiments an oxidizing solution may be added to the acid such as a concentration of hydrogen peroxide.

In another embodiment, the reactant may be a super-saturated solution of sodium acetate and the catalyst may be a nucleating stimulus such as an enclosed metal “clicker,” or small metal disk bent to be clicked back and forth like a button. The clicking may provide mechanical nucleation within the solution and trigger crystallization.

In some embodiments, the reactant may be a blend of magnesium powder, iron powder, sodium chloride, and an inert filler, and the catalyst may be water.

In a method of operation a warming pouch can be provided and pressure is applied to the warming device to burst an interior burst pouch 300 thereby releasing a catalyst into a reactant. The pouch may be massaged 305 to evenly distribute and mix the catalyst and reactant to generate a desired heat and form a putty consistency. Anti-fogging solution may then be applied to a sorbent pad adhered or otherwise integrated into the device 310. Anti-fogging solutions may comprise generally water, alcohol and surfactants. As discussed above, the sorbent pad may be colored a reference-white. An endoscope tip including a lens may then be brought in contact with the pad 315 and the pouch is then deformed to fit the shape of the endoscope 320. The endoscope is further held in place while absorbing heat transfer from the pouch 325 and optionally and simultaneously, conducting white-balancing through the lens of endoscope camera 335 and applying an anti-fogging treatment to the lens 330. Alternatively, the white-balancing and anti-fogging steps can be performed sequentially. Then the endoscope preparation is complete and the scope is ready for use in the body 340. Although the method has been described for preparing an endoscope, the warming device may be used to treat other medical devices or non-medical devices having lenses.

### Additional Embodiments

The following exemplary embodiments are provided, the numbering of which is not to be construed as designating levels of importance:

Embodiment 1 provides flexible warming device for generating and transferring heat to an endoscope including a sealed pouch forming an interior lumen enclosing an exothermic reactant, a burstable pouch positioned within the interior lumen of the sealed pouch containing a catalyst, which when mixed with the exothermic reactant generates heat, and at least one pouch surface configured to transfer the generated heat within the pouch to an endoscope positioned exterior to and contacting pouch, to absorb anti-fogging solution and transfer it to the endoscope, and provide a white-balancing color reference surface to a camera of the endoscope.

Embodiment 2 provides the flexible warming device of Embodiment 1, wherein the reactant comprises calcium oxide and the catalyst comprises an aqueous solution.

Embodiment 3 provides the flexible warming device of any of Embodiments 1-2, wherein the sealed pouch further comprises a rheological modifier configured to control the rate of heat generation when the catalyst mixes with the exothermic reactant.

Embodiment 4 provides the flexible warming device of any of Embodiments 1-3, wherein the exothermic reactant does not produce a gaseous byproduct or a toxic product in response to mixing with the catalyst.

Embodiment 5 provides the flexible warming device of any of Embodiments 1-4, wherein the exothermic reactant comprises a powder mixture.

Embodiment 6 provides the flexible warming device of any of Embodiments 1-5, wherein the powder mixture comprises a primary fast-reactant and a secondary reactant, wherein the secondary reactant reacts at a slower rate and reaches a peak heat generation with the catalyst later in time than the primary fast-reactant.

Embodiment 7 provides the flexible warming device of any of Embodiments 1-6, wherein the primary fast-reactant and secondary reactant react with the catalyst to generate a heat output in a range of 105-180 degrees Fahrenheit.

Embodiment 8 provides the flexible warming device of any of Embodiments 1-7, wherein the wherein the primary fast-reactant and secondary reactant react with the catalyst to generate a heat output in a range of 105-180 degrees Fahrenheit and maintain the heat output in the range for 20-30 minutes.

Embodiment 9 provides the flexible warming device of any of Embodiments 1-8, wherein the at least one pouch surface comprises a rubber material, a sorptive material, and the color-reference material is a white-balancing material.

Embodiment 10 provides the flexible warming device of any of Embodiments 1-9, wherein the at least one surface is a single surface on the device comprising heat-transferring, sorptive, and color-reference material properties.

Embodiment 11 provides the flexible warming device of any of Embodiments 1-10, wherein the at least one surface comprises three distinct surfaces spaced from each other along the outside of the device.

Embodiment 12 provides a device for warming an endoscope for insertion into a body including a flexible and malleable pouch having a single sealed interior lumen, a dry mixture containing an exothermic reactant positioned in the interior lumen, and a burstable pouch positioned in the interior lumen containing an aqueous catalyst, wherein the burstable pouch is configured to break in response to application of an external force to the flexible and malleable pouch and release the aqueous catalyst to mix with the dry mixture and generate heat.

Embodiment 13 provides the device for warming an endoscope for insertion into a body of Embodiment 12, wherein the exothermic reactant comprises calcium oxide and the catalyst comprises an aqueous solution.

Embodiment 14 provides the device for warming an endoscope for insertion into a body of any of Embodiments 12-13, wherein the dry mixture comprises a primary fast-reactant and a secondary reactant, wherein the secondary reactant reacts at a slower rate with the catalyst and reaches a maximum temperature later in time than the primary fast-reactant.

Embodiment 15 provides the device for warming an endoscope for insertion into a body of any of Embodiments 12-14, wherein the primary fast-reactant and secondary reactant react with the catalyst to generate a heat output in a range of 105-180 degrees Fahrenheit.

Embodiment 16 provides the device for warming an endoscope for insertion into a body of any of Embodiments 12-15, wherein the primary fast-reactant and secondary reactant react with the catalyst to generate a heat output in a range of 105-180 degrees Fahrenheit and maintain the heat output in the range for 20-30 minutes.

Embodiment 17 provides a method for preparing an endoscope for use inside the body including applying a force to a flexible warming device to release a catalyst from a rupturable pouch therein, massaging the flexible warming device to distribute the catalyst with a reactant in the flexible warming device thereby generating heat, and molding the flexible warming device to contact the endoscope, thereby transferring heat to the endoscope and providing contact between a lens of the endoscope and a color-reference surface of the endoscope.

Embodiment 18 provides the method of Embodiment 17 further including applying an anti-fogging solution to the color-reference surface of the device and transferring the anti-fogging solution to the lens via the contact between the lens and the color-reference surface, wherein the color reference surface is a sorptive surface.

Embodiment 19 provides the method of any of Embodiments 17-18, further including white-balancing a camera of the endoscope using the color-reference surface, wherein the heat transferring, anti-fogging solution transferring, and white-balancing are performed simultaneously.

Embodiment 20 provides the method of any of Embodiments 17-19, wherein the catalyst comprises an aqueous solution and the reactant comprises a mixture of fast and slow-reacting powders.

Embodiment 21 provides a warming device mixture including a reactant mixture comprising a primary fast reactant and a secondary, relatively slower reactant, and a catalyst comprising a kosmotropic agent in an aqueous solution, wherein the catalyst and the reactant are configured to generate heat output in a range of 105-180 degrees Fahrenheit and maintain the heat output in the range for 20-30 minutes.

Embodiment 22 provides the warming device of Embodiment 21, wherein the primary fast-reactant comprises calcium chloride, the secondary reactant comprises calcium oxide, and the kosmotropic agent comprises glycerol.

Embodiment 23 provides the warming device of any of Embodiments 21-22, wherein the primary fast-reactant comprises calcium chloride, the secondary reactant comprises calcium oxide, and the kosmotropic agent comprises propylene glycol.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

What is claimed is:

1. A flexible warming device for generating and transferring heat to an endoscope comprising:
a sealed pouch forming an interior lumen enclosing an exothermic reactant,
a burstable pouch positioned within the interior lumen of the sealed pouch containing a catalyst, which when mixed with the exothermic reactant generates heat, and
at least one pouch surface configured to transfer the generated heat within the pouch to an endoscope positioned exterior to and contacting pouch, to absorb anti-fogging solution and transfer it to the endoscope, and provide a white-balancing color reference surface to a camera of the endoscope.

2. The flexible warming device of claim 1, wherein the reactant comprises calcium oxide and the catalyst comprises an aqueous solution.

3. The flexible warming device of claim 2, wherein the sealed pouch further comprises a rheological modifier configured to control the rate of heat generation when the catalyst mixes with the exothermic reactant.

4. The warming device of claim 1, wherein the exothermic reactant does not produce a gaseous byproduct or a toxic product in response to mixing with the catalyst.

5. The warming device of claim 1, wherein the exothermic reactant comprises a powder mixture.

6. The warming device of claim 5, wherein the powder mixture comprises a primary fast-reactant and a secondary reactant, wherein the secondary reactant reacts at a slower rate and reaches a peak heat generation with the catalyst later in time than the primary fast-reactant.

7. The warming device of claim 6, wherein the primary fast-reactant and secondary reactant react with the catalyst to generate a heat output in a range of 105-180 degrees Fahrenheit.

8. The warming device of claim 6, wherein the wherein the primary fast-reactant and secondary reactant react with the catalyst to generate a heat output in a range of 105-180 degrees Fahrenheit and maintain the heat output in the range for 20-30 minutes.

9. The warming device of claim 1, wherein the at least one pouch surface comprises a rubber material, a sorptive material, and the color-reference material is a white-balancing material.

10. The warming device of claim 9, wherein the at least one surface is a single surface on the device comprising heat-transferring, sorptive, and color-reference material properties.

11. The warming device of claim 9, wherein the at least one surface comprises three distinct surfaces spaced from each other along the outside of the device.

12. A device for warming an endoscope for insertion into a body, the device comprising:
a flexible and malleable pouch having a single sealed interior lumen,
a dry mixture containing an exothermic reactant positioned in the interior lumen, and
a burstable pouch positioned in the interior lumen containing an aqueous catalyst,
wherein the burstable pouch is configured to break in response to application of an external force to the flexible and malleable pouch and release the aqueous catalyst to mix with the dry mixture and generate heat.

13. The device of claim 12, wherein the exothermic reactant comprises calcium oxide and the catalyst comprises an aqueous solution.

14. The device of claim 13, wherein the dry mixture comprises a primary fast-reactant and a secondary reactant, wherein the secondary reactant reacts at a slower rate with the catalyst and reaches a maximum temperature later in time than the primary fast-reactant.

15. The device of claim 14, wherein the primary fast-reactant and secondary reactant react with the catalyst to generate a heat output in a range of 105-180 degrees Fahrenheit.

16. The device of claim 14, wherein the primary fast-reactant and secondary reactant react with the catalyst to generate a heat output in a range of 105-180 degrees Fahrenheit and maintain the heat output in the range for 20-30 minutes.

17. A method for preparing an endoscope for use inside the body comprising:
applying a force to a flexible warming device to release a catalyst from a rupturable pouch therein,
massaging the flexible warming device to distribute the catalyst with a reactant in the flexible warming device thereby generating heat, and
molding the flexible warming device to contact the endoscope, thereby transferring heat to the endoscope and providing contact between a lens of the endoscope and a color-reference surface of the endoscope.

18. The method of claim 17, further comprising applying an anti-fogging solution to the color-reference surface of the device and transferring the anti-fogging solution to the lens via the contact between the lens and the color-reference surface, wherein the color reference surface is a sorptive surface.

19. The method of claim 18, further comprising white-balancing a camera of the endoscope using the color-reference surface, wherein the heat transferring, anti-fogging solution transferring, and white-balancing are performed simultaneously.

20. The method of claim 17, wherein the catalyst comprises an aqueous solution and the reactant comprises a mixture of fast and slow-reacting powders.

21. A warming device mixture comprising:
a reactant mixture comprising a primary fast reactant and a secondary, relatively slower reactant, and
a catalyst comprising a kosmotropic agent in an aqueous solution,
wherein the catalyst and the reactant are configured to generate heat output in a range of 105-180 degrees Fahrenheit and maintain the heat output in the range for 20-30 minutes.

22. The warming device mixture of claim 21, wherein the primary fast-reactant comprises calcium chloride, the secondary reactant comprises calcium oxide, and the kosmotropic agent comprises glycerol.

23. The warming device mixture of claim 21, wherein the primary fast-reactant comprises calcium chloride, the secondary reactant comprises calcium oxide, and the kosmotropic agent comprises propylene glycol.
